(19) United States
(12) Patent Application Publication
(10) Pub. No.: US 2020/0294618 A1
(43) Pub. Date: Sep. 17, 2020

(54) METHOD TO PERFORM MEDICAL PROCEDURES ON BREAST CANCER PATIENTS GUIDED BY AN SNP DERIVED POLYGENIC RISK SCORE

(71) Applicant: Ambry Genetics Corporation, Aliso Viejo, CA (US)

(72) Inventors: Mary Helen Black, Aliso Viejo, CA (US); Shuwei Li, Aliso Viejo, CA (US); Holly LaDuca, Aliso Viejo, CA (US); Hsiao-Mei Lu, Aliso Viejo, CA (US); AJ Stuenkel, Aliso Viejo, CA (US); Chia-Ling Gau, Aliso Viejo, CA (US); Jessica Profato, Aliso Viejo, CA (US)

(21) Appl. No.: 16/351,378

(22) Filed: Mar. 12, 2019

(51) Int. Cl. G16B 20/20; C12Q 1/6886

(52) U.S. Cl. CPC G16B 20/20; C12Q 2600/118; C12Q 2600/156; C12Q 1/6886

(57) ABSTRACT

Disclosed herein are methods for performing a medical procedure on patients by determining the probability a patient will develop breast cancer through the use of a polygenic risk score that uses single nucleotide polymorphisms in its calculation. Also disclosed herein is a method for diagnosing patients with having an increased risk for the development of breast cancer, that is based on using a polygenic risk score derived from single nucleotide polymorphisms. In particular, as disclosed herein is a unique set of single nucleotide polymorphism with which to calculate the polygenic risk score.

### FIELD

The methods relate to performing a medical procedure on patients diagnosed with having an increased risk for the development of breast cancer, that is based on using a polygenic risk score derived from single nucleotide polymorphisms. The methods further relates to a method for diagnosing patients with having an increased risk for the development of breast cancer, that is based on using a polygenic risk score derived from single nucleotide polymorphisms. The invention further relates to a unique set of single nucleotide polymorphisms for use in deriving the polygenic risk score.

### BACKGROUND

Breast cancer is the most common cancer affecting women in the world. It is estimated that worldwide over 500,000 women died in 2011 due to breast cancer (Global Health Estimates, WHO 2013).

Breast cancer survival rates vary greatly worldwide. The survival rate can range from 80% in developed countries to below 40% in developing countries (Coleman et al., 2008). Early detecting in conjunction with various screening methods can potentially decrease the mortality associated with breast cancer.

Genome-wide association studies (GWAS) are observational studies of a set of genetic variants in individuals to see if any variant is associated with a particular trait. GWASs typically focus on associations between single-nucleotide polymorphisms (SNPs) and human diseases. In contrast to testing a small number of genetic regions, GWASs analyze the entire genome.

Since 2007, GWASs have identified many common SNPs, each with a modest contribution to breast cancer risk (Easton, D. F., et al., 2007).

As these SNPs are associated with relative risks ranging from 1.03-1.41 (Michailidou, K., et al., 2017), no individual SNP is usually informative on its own. However, a score based on combined genotypes across a large number of SNPs may have substantial predictive value for risk stratification (Mavaddat, N., et al., 2015; Dite, G. S., et al., 2016; Mealiffe, M. E., et al., 2010; Reeves, G. K., 2010; Shieh, Y., et al., 2016). While the utility of such a score has been investigated in large studies conducted in the general population, few have assessed its performance in high-risk women referred for genetic testing for breast cancer (Li, H., et al., 2017; Sawyer, S., et al., 2012).

SNP-based scores may have clinically useful predictive power in women referred for genetic testing due to a family history of disease. Sawyer et al. (2012) examined a 22-SNP polygenic risk score (PRS) comparing women who were diagnosed with breast cancer, who were either BRCA1/2 carriers or BRCA1/2 negative, to a set of controls. They found that BRCA1/2 negative cases had a significantly higher PRS than BRCA1/2 carriers or controls, and that BRCA1/2 negative cases in the highest quartile of the PRS distribution were more likely to have had early-onset breast cancer (<30 years of age) compared to those with a score in the lowest PRS quartile. Li et al. assessed a 24-SNP PRS among unaffected women from two familial breast cancer cohorts, and observed that women in the highest quintile of the PRS distribution were more than three times as likely to develop breast cancer as those in the lowest quintile (Li, H., et al., 2017).

Taken together, the data suggested that a SNP-based PRS may be useful for risk stratification in women with family history of breast cancer who are negative for high-penetrance breast cancer-susceptibility genes.

### SUMMARY

The present disclosure provides a method for performing a medical procedure by determining whether an individual has an increased risk for the development of breast cancer. The present disclosure also provides a method for diagnosis by determining whether an individual has an increased risk for the development of breast cancer. This disclosure sets forth processes, in addition to making and using the same, and other solutions to problems in the relevant field.

In some embodiments, there is provided a method for performing a medical procedure on a patient with a potential pre-disposition to cancer comprising: obtaining a nucleic acid sample from a patient, assaying the nucleic acid sample obtained from the patient for at least 50 single nucleotide polymorphisms (SNPs) set forth in Table 1, wherein for each SNP in this step, one or more of the following is assayed: the SNP from Table 1, another SNP located within 250 kilobases of the SNP from Table 1, and another SNP that has a pairwise r²=1.0 with the SNP from Table 1; calculating a polygenic risk score (PRS) based on the presence or absence of the at least 50 single nucleotide polymorphisms, wherein the polygenic risk score indicates a risk, relative to an average population, that the subject will develop breast cancer; and performing a medical procedure for the patient based on the PRS.

In some embodiments, there is provided a method for diagnosing a patient with a potential pre-disposition to cancer comprising: obtaining a nucleic acid sample from a patient, assaying the nucleic acid sample obtained from the patient for at least 50 single nucleotide polymorphisms (SNPs) set forth in Table 1, wherein for each SNP in this step, one or more of the following is assayed: the SNP from Table 1, another SNP located within 250 kilobases of the SNP from Table 1, and another SNP that has a pairwise r²=1.0 with the SNP from Table 1; calculating a polygenic risk score (PRS) based on the presence or absence of the at least 50 single nucleotide polymorphisms, wherein the polygenic risk score indicates a risk, relative to an average population, that the subject will develop breast cancer.

### BRIEF DESCRIPTIONS OF THE DRAWINGS

FIG. 1. Distribution of the sum of risk alleles across 100 SNPs, for cases compared to controls. The probability density on the y-axis represents the proportion of cases and controls, respectively, with a given risk allele count on the x-axis.

FIG. 2. The per allele odds ratio (95% CI) for breast cancer per quartile of PRS estimated in the case/control set compared to those reported by Shieh et al. 2016.

FIG. 3. The area under the receiver operating curve (AUROC) shows the accuracy of the PRS in distinguishing between breast cancer cases and controls.

### DETAILED DESCRIPTION

The following description is presented to enable one of ordinary skill in the art to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented, but is to be accorded the widest scope consistent with the principles and novel features disclosed herein.

### Definitions

As used herein, the term “biological sample,” refers to a sample derived from, obtained by, generated from, provided from, take from, or removed from an organism; or from fluid or tissue from the organism. Biological samples include, but are not limited to synovial fluid, whole blood, blood serum, blood plasma, urine, sputum, tissue, saliva, tears, spinal fluid, tissue section(s) obtained by biopsy, cell(s) that are placed in or adapted to tissue culture, sweat, mucous, fecal material, gastric fluid, abdominal fluid, amniotic fluid, cyst fluid, peritoneal fluid, pancreatic juice, breast milk, lung lavage, marrow, gastric acid, bile, semen, pus, aqueous humor, transudate, and the like including derivatives, portions and combinations of the foregoing. In some examples, biological samples include, but are not limited, to blood and/or plasma. In some examples, biological samples include, but are not limited, to urine or stool. Biological samples include, but are not limited, to saliva. Biological samples include, but are not limited, to tissue dissections and tissue biopsies. Biological samples include, but are not limited, samples that can provide nucleic acids for analysis. Biological samples include, but are not limited, any derivative or fraction of the aforementioned biological samples.

As used herein, the term “patient” refers to a human female subject. The methods and uses of the invention described herein are useful to treat a human.

As used herein, the term “Ashkenazi Jew” refers to a population whose recent ancestry over the past millennium traces to Central and Eastern Europe.

As used herein, the term “Caucasian” refers to individuals whose recent ancestry over the past millennium traces to Northern Europe.

As used herein, the term “Northern Europe” is the general term for the geographical region in Europe that is North of the Baltic Sea and includes the British Isles, Greenland, Sweden, Norway, Lithuania, Latvia, Estonia, and Finland.

As used herein, the term “single nucleotide polymorphism” or “SNP” refers to a genetic variation between individuals wherein the variation is a single nitrogenous base position in the DNA of organisms that is variable. In other words, an SNP refers to a polymorphism at a single nucleotide position in a genome where the nucleotide at the specified position varies between individuals or populations.

As used herein, the term, “SNPs” is the plural of SNP.

As used herein, the term “allele frequency” (p) refers to the relative frequency at which an allele is present at a locus within a population expressed as a fraction or percentage. For example, for a given allele “A”, individuals who are diploid may have the following genotypes: “AA”, “Aa” or “aa”. The genotype frequencies for an allele “A” are calculated by multiplying the number of individuals who have the genotypes: “AA”, “Aa” or “aa” by 2, 1, or 0, respectively to determine how many alleles for “A” and “a” exist within the population. The allele frequency is calculated by dividing the total number of alleles “A” in a population by the total number of alleles.

As used herein, a “risk allele frequency” refers to the allele frequency of a risk allele. A risk allele is an allele that is associated with an increased risk of contracting a disease.

As used herein, the term per allele odds ratio (OR) is an odds ratio with respect to each copy of an allele. An allelic OR describes the association between disease and allele by comparing the odds of disease in an individual carrying allele “A” to the odds of disease in an individual carrying allele “a”. An OR of 1.0 means that the DNA variant has no affect on the odds of having the disease, while values above 1.0 indicate a statistical association between that variant and having the disease. OR values below 1 indicate a lower association of disease.

An individual has “triple negative breast cancer” if the individual has breast cancer that tests negative for estrogen receptors, progesterone receptors, and is not overexpressing the HER2 protein.

As used herein, the term “medical procedure” is also synonymous with treatment.

As used herein, the term “treatment” or “treating” means any treatment of a disease or condition in a subject, such as a human female subject, including for example: 1) preventing or protecting against the disease or condition, including, causing the clinical symptoms not to develop; 2) inhibiting the disease or condition, including, arresting or suppressing the development of clinical symptoms; and/or 3) relieving the disease or condition, including, causing the regression or elimination of clinical symptoms. Treating includes administering therapeutic agents to a subject in need thereof.

As used herein, the term “linkage disequilibrium” is the non-random association of alleles at different loci in a given population. Two or more alleles are said to be in linkage equilibrium when they occur randomly in a population. Two or more alleles are in linkage disequilibrium when they do not occur randomly with respect to each other.

As used herein, the term “pairwise r²” indicates the amount of linkage disequilibrium between two SNPs. An r²=1 indicates that the SNPs are in complete linkage disequilibrium.

In this disclosure, methods are presented demonstrating the effectiveness of a PRS, based on the combined effects of 100 SNPs previously reported in multiple large GWAS studies, in predicting breast cancer in high-risk women referred for genetic testing who tested negative for pathogenic or likely pathogenic variants in known breast cancer susceptibility genes.

The disclosure herein sets forth embodiments for performing a medical procedure on a patient based on calculating a polygenic risk score of the patient. The methods herein provide a polygenic risk score, based on a select number of single nucleotide polymorphisms as listed in Table 1, that indicates the potential of developing breast cancer in a patient.

The disclosure herein sets forth embodiments for diagnosing a patient based on calculating a polygenic risk score of the patient. The methods herein provide a polygenic risk score, based on a select number of single nucleotide polymorphisms as listed in Table 1, that indicates the potential of developing breast cancer in a patient.

TABLE 1
List of SNPs used in the calculation of PRS.

| | | | If Proxy, | | | |
|---|---|---|---|---|---|---|
| | | | LD | | | |
| | | | in EUR | Odds Ratio | Risk Allele | |
| | | Original/ | (distance | (OR) | Frequency | |
| SNP ID | Bp(GRCh37) | Proxy | in bp) | (95% CI) | (P) | Reference for OR |
| rs11249433 | 121280613 | Original | | 1.10(1.09-1.12) | 45.0% | Michailidou et al. |
| | | | | | | 2017 |
| rs11552449 | 114448389 | Original | | 1.06(1.04-1.07) | 18.5% | Michailidou et al. |
| | | | | | | 2017 |
| rs12048493 | 149927034 | Original | | 1.05(1.04-1.06) | 36.6% | Michailidou et al. |
| | | | | | | 2017 |
| rs12405132 | 145644984 | Original | | 1.04(1.03-1.05) | 64.9% | Michailidou et al. |
| | | | | | | 2017 |
| rs17489300 | 202179042 | Original | | 1.11(1.08-1.15) | 59.2% | Couch et al. 2016 |
| rs4245739 | 204518842 | Original | | 1.15(1.11-1.20) | 29.1% | Milne et al. 2017 |
| rs616488 | 10566215 | Original | | 1.06(1.04-1.09) | 67.4% | Michailidou et al. |
| | | | | | | 2017 |
| rs72755295 | 242034263 | Original | | 1.15(1.09-1.22) | 3.7% | Michailidou et al. |
| | | | | | | 2017 |
| rs11903787 | 121088182 | Original | | 1.05(1.03-1.06) | 73.4% | Michailidou et al. |
| | | | | | | 2017 |
| rs12710696 | 19320803 | Original | | 1.04(1.02-1.05) | 34.8% | Michailidou et al. |
| | | | | | | 2017 |
| rs13387042 | 217905832 | Original | | 1.13(1.12-1.14) | 50.7% | Michailidou et al. |
| | | | | | | 2017 |
| rs1550623 | 174212894 | Original | | 1.05(1.04-1.07) | 84.8% | Michailidou et al. |
| | | | | | | 2017 |
| rs2016394 | 172972971 | Original | | 1.04(1.03-1.06) | 55.8% | Michailidou et al. |
| | | | | | | 2017 |
| rs4849887 | 121245122 | Original | | 1.10(1.06-1.14) | 88.2% | Michailidou et al. |
| | | | | | | 2017 |
| rs67073037 | 29119585 | Original | | 1.09(1.05-1.14) | 80.0% | Couch et al. 2016 |
| rs1053338 | 63967900 | Original | | 1.06(1.04-1.08) | 16.0% | Michailidou et al. |
| | | | | | | 2017 |
| rs12493607 | 30682939 | Original | | 1.05(1.04-1.06) | 33.2% | Michailidou et al. |
| | | | | | | 2017 |
| rs4973768 | 27416013 | Original | | 1.10(1.08-1.12) | 49.9% | Michailidou et al. |
| | | | | | | 2017 |
| rs6762644 | 4742276 | Original | | 1.06(1.04-1.07) | 34.5% | Michailidou et al. |
| | | | | | | 2017 |
| rs6796502 | 46866866 | Original | | 1.09(1.05-1.12) | 91.0% | Michailidou et al. |
| | | | | | | 2017 |
| rs6828523 | 175846426 | Original | | 1.11(1.08-1.15) | 89.8% | Michailidou et al. |
| | | | | | | 2017 |
| rs9790517 | 106084778 | Original | | 1.05(1.03-1.08) | 21.0% | Michailidou et al. |
| | | | | | | 2017 |
| rs10472076 | 58184061 | Original | | 1.04(1.02-1.05) | 37.8% | Michailidou et al. |
| | | | | | | 2017 |
| rs10941679 | 44706498 | Original | | 1.14(1.12-1.15) | 23.0% | Michailidou et al. |
| | | | | | | 2017 |
| rs13162653 | 16187528 | Original | | 1.05(1.03-1.08) | 53.6% | Michailidou et al. |
| | | | | | | 2015 |
| rs1353747 | 58337481 | Original | | 1.06(1.04-1.09) | 91.2% | Michailidou et al. |
| | | | | | | 2017 |
| rs1432679 | 158244083 | Original | | 1.07(1.05-1.09) | 47.49% | Michailidou et al. |
| | | | | | | 2017 |
| rs2012709 | 32567732 | Original | | 1.04(1.02-1.05) | 44.99% | Michailidou et al. |
| | | | | | | 2017 |
| rs2736108 | 1297488 | Original | | 1.06(1.04-1.09) | 69.89% | Michailidou et al. |
| | | | | | | 2017 |
| rs3215401 | 1296255 | Original | | 1.07(1.05-1.08) | 68.9% | Michailidou et al. |
| | | | | | | 2017 |
| rs4415084 | 44662515 | Original | | 1.10(1.08-1.11) | 41.09% | Michailidou et al. |
| | | | | | | 2017 |
| rs7707921 | 81538046 | Original | | 1.05(1.04-1.07) | 76.2% | Michailidou et al. |
| | | | | | | 2017 |
| rs889312 | 56031884 | Original | | 1.13(1.11-1.14) | 29.0% | Michailidou et al. |
| | | | | | | 2017 |
| rs11242675 | 1318878 | Original | | 1.06(1.04-1.09) | 67.7% | Michailidou et al. |
| | | | | | | 2015 |
| rs12665607 | 151946629 | Original | | 1.17(1.15-1.20) | 9.1% | Michailidou et al. |
| | | | | | | 2017 |
| rs17529111 | 82128386 | Original | | 1.05(1.03-1.06) | 21.3% | Michailidou et al. |
| | | | | | | 2017 |
| rs204247 | 13722523 | Original | | 1.05(1.03-1.07) | 43.0% | Michailidou et al. |
| | | | | | | 2017 |
| rs2046210 | 151948366 | Original | | 1.09(1.07-1.10) | 34.6% | Michailidou et al. |
| | | | | | | 2017 |
| rs2180341 | 127600630 | Original | | 1.41(1.25-1.59) | 28.7% | Gold et al. 2008 |
| rs910416 | 152432902 | Proxy (for | 1.0 (4114) | 1.07(1.06-1.08) | 50.5% | Michailidou et al. |
| | | rs2747652) | | | | 2017 |
| rs9257408 | 28926220 | Original | | 1.03(1.02-1.05) | 44.3% | Michailidou et al. |
| | | | | | | 2017 |
| rs9397437 | 151952332 | Original | | 1.20(1.17-1.23) | 8.2% | Michailidou et al. |
| | | | | | | 2017 |
| rs4593472 | 130667121 | Original | | 1.04(1.03-1.06) | 65.8% | Michailidou et al. |
| | | | | | | 2017 |
| rs6964587 | 91630620 | Original | | 1.04(1.03-1.05) | 40.4% | Michailidou et al. |
| | | | | | | 2017 |
| rs720475 | 144074929 | Original | | 1.05(1.04-1.06) | 71.8% | Michailidou et al. |
| | | | | | | 2017 |
| rs11780156 | 129194641 | Original | | 1.06(1.05-1.08) | 20.1% | Michailidou et al. |
| | | | | | | 2017 |
| rs13267382 | 117209548 | Original | | 1.04(1.03-1.06) | 33.6% | Michailidou et al. |
| | | | | | | 2017 |
| rs13281615 | 128355618 | Original | | 1.11(1.09-1.12) | 46.5% | Michailidou et al. |
| | | | | | | 2017 |
| rs13365225 | 36858483 | Original | | 1.08(1.06-1.10) | 84.5% | Michailidou et al. |
| | | | | | | 2017 |
| rs1562430 | 128387852 | Original | | 1.11(1.09-1.12) | 60.1% | Michailidou et al. |
| | | | | | | 2017 |
| rs2943559 | 76417937 | Original | | 1.12(1.10-1.15) | 7.6% | Michailidou et al. |
| | | | | | | 2017 |
| rs6472903 | 76230301 | Original | | 1.08(1.06-1.10) | 84.5% | Michailidou et al. |
| | | | | | | 2017 |
| rs9693444 | 29509616 | Original | | 1.06(1.05-1.08) | 35.1% | Michailidou et al. |
| | | | | | | 2017 |
| rs10759243 | 110306115 | Original | | 1.06(1.05-1.08) | 29.7% | Michailidou et al. |
| | | | | | | 2017 |
| rs865686 | 110888478 | Original | | 1.10(1.09-1.12) | 62.7% | Michailidou et al. |
| | | | | | | 2017 |
| rs10995190 | 64278682 | Original | | 1.14(1.12-1.16) | 84.8% | Michailidou et al. |
| | | | | | | 2017 |
| rs11199914 | 123093901 | Original | | 1.05(1.03-1.08) | 68.7% | Michailidou et al. |
| | | | | | | 2017 |
| rs11814448 | 22315843 | Original | | 1.20(1.15-1.25) | 2.1% | Michailidou et al. |
| | | | | | | 2017 |
| rs2981579 | 123337335 | Original | | 1.27(1.24-1.29) | 43.8% | Michailidou et al. |
| | | | | | | 2017 |
| rs704010 | 80841148 | Original | | 1.08(1.06-1.10) | 43.2% | Michailidou et al. |
| | | | | | | 2017 |
| rs7072776 | 22032942 | Original | | 1.06(1.05-1.08) | 29.7% | Michailidou et al. |
| | | | | | | 2017 |
| rs7904519 | 114773927 | Original | | 1.05(1.03-1.07) | 50.9% | Michailidou et al. |
| | | | | | | 2017 |
| rs3817198 | 1909006 | Original | | 1.06(1.05-1.07) | 32.3% | Michailidou et al. |
| | | | | | | 2017 |
| rs3903072 | 65583066 | Original | | 1.04(1.03-1.06) | 6% | Michailidou et al. |
| | | | | | | 2017 |
| rs554219 | 69331642 | Original | | 1.26(1.23-1.30) | 12.0% | Michailidou et al. |
| | | | | | | 2015 |
| rs745382 | 129462233 | Proxy (for | 1.0 (1062) | 1.05(1.03-1.08) | 56.7% | Michailidou et al. |
| | | rs11820646) | | | | 2017 |
| rs78540526 | 69331418 | Original | | 1.32(1.29-1.35) | 7.2% | Michailidou et al. |
| | | | | | | 2017 |
| rs10771399 | 28155080 | Original | | 1.16(1.12-1.20) | 89.3% | Michailidou et al. |
| | | | | | | 2015 |
| rs12422552 | 14413931 | Original | | 1.04(1.02-1.07) | 29.6% | Michailidou et al. |
| | | | | | | 2015 |
| rs1292011 | 115836522 | Original | | 1.09(1.06-1.11) | 58.8% | Michailidou et al. |
| | | | | | | 2017 |
| rs17356907 | 96027759 | Original | | 1.10(1.08-1.11) | 71.6% | Michailidou et al. |
| | | | | | | 2017 |
| rs7297051 | 28174817 | Original | | 1.16(1.12-1.20) | 77.5% | Michailidou et al. |
| | | | | | | 2017 |
| rs11571833 | 32972626 | Original | | 1.31(1.23-1.41) | 1.0% | Michailidou et al. |
| | | | | | | 2017 |
| rs17181761 | 73811471 | Original | | 1.04(1.03-1.05) | 28.7% | Michailidou et al. |
| | | | | | | 2017 |
| rs6562760 | 73957681 | Original | | 1.05(1.03-1.06) | 75.2% | Michailidou et al. |
| | | | | | | 2017 |
| rs11627032 | 93104072 | Original | | 1.05(1.03-1.06) | 74.3% | Michailidou et al. |
| | | | | | | 2017 |
| rs2236007 | 37132769 | Original | | 1.07(1.06-1.09) | 77.0% | Michailidou et al. |
| | | | | | | 2017 |
| rs2588809 | 68660428 | Original | | 1.06(1.05-1.08) | 20.0% | Michailidou et al. |
| | | | | | | 2017 |
| rs941764 | 91841069 | Original | | 1.05(1.03-1.06) | 35.8% | Michailidou et al. |
| | | | | | | 2017 |
| rs999737 | 69034682 | Original | | 1.10(1.09-1.12) | 77.1% | Michailidou et al. |
| | | | | | | 2017 |
| rs11075995 | 53855291 | Original | | 1.04(1.03-1.06) | 19.3% | Michailidou et al. |
| | | | | | | 2017 |
| rs13329835 | 80650805 | Original | | 1.08(1.06-1.11) | 22.8% | Michailidou et al. |
| | | | | | | 2017 |
| rs17817449 | 53813367 | Original | | 1.06(1.05-1.07) | 58.0% | Michailidou et al. |
| | | | | | | 2017 |
| rs3803662 | 52586341 | Original | | 1.23(1.21-1.24) | 26.7% | Michailidou et al. |
| | | | | | | 2017 |
| rs8051542 | 52534167 | Original | | 1.09(1.06-1.13) | 40.9% | Michailidou et al. |
| | | | | | | 2017 |
| rs146699004 | 29230520 | Original | | 1.08(1.04-1.10) | 71.0% | Michailidou et al. |
| | | | | | | 2017 |
| rs6504950 | 53056471 | Original | | 1.07(1.06-1.08) | 73.1% | Michailidou et al. |
| | | | | | | 2017 |
| rs745570 | 77781725 | Original | | 1.04(1.03-1.05) | 52.09% | Michailidou et al. |
| | | | | | | 2017 |
| rs1436904 | 24570667 | Original | | 1.05(1.04-1.06) | 57.0% | Michailidou et al. |
| | | | | | | 2017 |
| rs1667550 | 24332476 | Proxy (for | 0.9 (4948) | 1.11(1.08-1.16) | 65.9% | Michailidou et al. |
| | | rs527616) | | | | 2015 |
| rs6507583 | 42399590 | Original | | 1.09(1.06-1.12) | 93.3% | Michailidou et al. |
| | | | | | | 2017 |
| rs3760982 | 44286513 | Original | | 1.05(1.03-1.07) | 47.5% | Michailidou et al. |
| | | | | | | 2017 |
| rs4808801 | 18571141 | Original | | 1.07(1.06-1.09) | 65.9% | Michailidou et al. |
| | | | | | | 2017 |
| rs56069439 | 17393925 | Original | | 1.04(1.03-1.05) | 27.0% | Michailidou et al. |
| | | | | | | 2017 |
| rs16991615 | 5948227 | Original | | 1.08(1.05-1.11) | 7.8% | Michailidou et al. |
| | | | | | | 2017 |
| rs2823093 | 16520832 | Original | | 1.07(1.05-1.08) | 73.0% | Michailidou et al. |
| | | | | | | 2017 |
| rs132390 | 29621477 | Original | | 1.10(1.06-1.14) | 2.0% | Michailidou et al. |
| | | | | | | 2017 |
| rs17001868 | 40778231 | Original | | 1.10(1.08-1.13) | 9.8% | Michailidou et al. |
| | | | | | | 2017 |
| rs17879961 | 29121087 | Original | | 1.28(1.17-1.39) | 0.0% | Michailidou et al. |
| | | | | | | 2017 |
| rs73167067 | 40875199 | Proxy (for | 1.0 (1035) | 1.10(1.08-1.13) | 9.0% | Michailidou et al. |
| | | rs6001930) | | | | 2015 |

In some embodiments the minimum number of SNPs in Table 1 used to calculate the PRS are: 50, 55, 60, 65, 70, 75, 80, 81, 82, 83, 84, 85, 86, 87, 88, 89, 90, 91, 92, 93, 94, 95, 96, 97, 98, 99, or 100.

In some embodiments, at least 50 of the single nucleotide polymorphisms as set forth in Table 1 are used to calculate the PRS. In some embodiments, at least 55 of the single nucleotide polymorphisms as set forth in Table 1 are used to calculate the PRS. In some embodiments, at least 60 of the single nucleotide polymorphisms as set forth in Table 1 are used to calculate the PRS. In some embodiments, at least 65 of the single nucleotide polymorphisms as set forth in Table 1 are used to calculate the PRS. In other embodiments, at least 70 of the single nucleotide polymorphisms as set forth in Table 1 are used to calculate the PRS. In other embodiments, at least 75 of the single nucleotide polymorphisms as set forth in Table 1 are used to calculate the PRS. In other embodiments, at least 80 of the single nucleotide polymorphisms as set forth in Table 1 are used to calculate the PRS. In other embodiments, at least 81 of the single nucleotide polymorphisms as set forth in Table 1 are used to calculate the PRS. In other embodiments, at least 82 of the single nucleotide polymorphisms as set forth in Table 1 are used to calculate the PRS. In other embodiments, at least 83 of the single nucleotide polymorphisms as set forth in Table 1 are used to calculate the PRS. In other embodiments, at least 84 of the single nucleotide polymorphisms as set forth in Table 1 are used to calculate the PRS. In other embodiments, at least 85 of the single nucleotide polymorphisms as set forth in Table 1 are used to calculate the PRS. In other embodiments, at least 86 of the single nucleotide polymorphisms as set forth in Table 1 are used to calculate the PRS. In other embodiments, at least 87 of the single nucleotide polymorphisms as set forth in Table 1 are used to calculate the PRS. In other embodiments, at least 88 of the single nucleotide polymorphisms as set forth in Table 1 are used to calculate the PRS. In other embodiments, at least 89 of the single nucleotide polymorphisms as set forth in Table 1 are used to calculate the PRS. In other embodiments, at least 90 of the single nucleotide polymorphisms as set forth in Table 1 are used to calculate the PRS. In other embodiments, at least 91 of the single nucleotide polymorphisms as set forth in Table 1 are used to calculate the PRS. In other embodiments, at least 92 of the single nucleotide polymorphisms as set forth in Table 1 are used to calculate the PRS. In other embodiments, at least 93 of the single nucleotide polymorphisms as set forth in Table 1 are used to calculate the PRS. In other embodiments, at least 94 of the single nucleotide polymorphisms as set forth in Table 1 are used to calculate the PRS. In other embodiments, at least 95 of the single nucleotide polymorphisms as set forth in Table 1 are used to calculate the PRS. In other embodiments, at least 96 of the single nucleotide polymorphisms as set forth in Table 1 are used to calculate the PRS. In other embodiments, at least 97 of the single nucleotide polymorphisms as set forth in Table 1 are used to calculate the PRS. In other embodiments, at least 98 of the single nucleotide polymorphisms as set forth in Table 1 are used to calculate the PRS. In other embodiments, at least 99 of the single nucleotide polymorphisms as set forth in Table 1 are used to calculate the PRS. In other embodiments, all of the single nucleotide polymorphisms as set forth in Table 1 are used to calculate the PRS.

In some embodiments, the 50 SNPs used to calculate the PRS, as set forth in Table 1, are chosen in descending order with respect to the odds ratio. In some embodiments, SNPs with an odds ratio of 1.07 or more, as set forth in Table 1, are selected to calculate the PRS. In some embodiments, SNPs with an odds ratio of 1.08 or more, as set forth in Table 1, are selected to calculate the PRS. In some embodiments, SNPs with an odds ratio of 1.09 or more, as set forth in Table 1, are selected to calculate the PRS. In some embodiments, SNPs with an odds ratio of 1.10 or more, as set forth in Table 1, are selected to calculate the PRS.

In some embodiments, the SNPs of Table 2 may be used as a proxy for the SNPs of Table 1. Table 2 lists SNPs that are within 250 kilobases of the SNPs in Table 1 and have a pairwise r²=1.0. Table 2 also lists SNPs that are present in Table 1 by either 1 (indicating yes) or 0 (indicating no).

TABLE 2
List of Proxy SNPs that may
be used in the calculation of PRS.

| | | Table | | | |
|---|---|---|---|---|---|
| | | 1 | | | |
| | | SNP | | | |
| | bp | (pres- | | | |
| Chr | (GRCh37) | ent) | rsID | Ref | Alt |
| 1 | 10566215 | 1 | rs616488 | A | G |
| 1 | 114445880 | 0 | rs7513707 | G | A |
| 1 | 114448389 | 1 | rs11552449 | C | G, T |
| 1 | 121280613 | 1 | rs11249433 | A | G |
| 1 | 145644984 | 1 | rs12405132 | C | T |
| 1 | 149927034 | 1 | rs12048493 | A | C |
| 1 | 202179042 | 1 | rs17489300 | A | C |
| 1 | 202180401 | 0 | rs12033508 | A | G |
| 1 | 204518842 | 1 | rs4245739 | C | A |
| 1 | 242034263 | 1 | rs72755295 | A | G |
| 2 | 19320803 | 1 | rs12710696 | T | C |
| 2 | 19322004 | 0 | rs6731836 | C | A |
| 2 | 19322818 | 0 | rs13420196 | A | C |
| 2 | 29119585 | 1 | rs67073037 | A | T |
| 2 | 121088182 | 1 | rs11903787 | G | A |
| 2 | 121242996 | 0 | rs4848600 | C | T |
| 2 | 121243011 | 0 | rs4848601 | G | C |
| 2 | 121243108 | 0 | rs4848602 | C | T |
| 2 | 121243112 | 0 | rs4848603 | C | T |
| 2 | 121243478 | 0 | rs4849881 | C | A |
| 2 | 121243526 | 0 | rs7598664 | C | T |
| 2 | 121243690 | 0 | rs7571527 | G | A |
| 2 | 121243713 | 0 | rs4849882 | C | A |
| 2 | 121244132 | 0 | rs12711945 | G | A |
| 2 | 121244172 | 0 | rs12711946 | G | A |
| 2 | 121244272 | 0 | rs12711947 | T | C |
| 2 | 121244492 | 0 | rs9308781 | G | A |
| 2 | 121244809 | 0 | rs9308782 | A | G |
| 2 | 121244905 | 0 | rs4849883 | C | T |
| 2 | 121244947 | 0 | rs4849884 | A | C |
| 2 | 121245080 | 0 | rs4849885 | T | C |
| 2 | 121245096 | 0 | rs4849886 | T | C |
| 2 | 121245122 | 1 | rs4849887 | T | C |
| 2 | 121245483 | 0 | rs71989020 | TGC | T |
| | | | | CTG | |
| 2 | 121245540 | 0 | rs12616822 | G | A |
| 2 | 121245603 | 0 | rs11123555 | T | C |
| 2 | 121245735 | 0 | rs10864991 | G | A |
| 2 | 121245996 | 0 | rs11123556 | A | G |
| 2 | 121246568 | 0 | rs10179592 | T | C |
| 2 | 172972971 | 1 | rs2016394 | G | A |
| 2 | 174212894 | 1 | rs1550623 | G | A |
| 2 | 174212910 | 0 | rs1550622 | A | G |
| 2 | 217905779 | 0 | rs13412666 | G | A |
| 2 | 217905832 | 1 | rs13387042 | A | G |
| 2 | 217906246 | 0 | rs13426489 | T | G |
| 3 | 4742251 | 0 | rs6762558 | A | G |
| 3 | 4742276 | 1 | rs6762644 | A | G |
| 3 | 4742779 | 0 | rs6774180 | G | A |
| 3 | 27416013 | 1 | rs4973768 | C | T |
| 3 | 30679970 | 0 | rs12495646 | C | A |
| 3 | 30682939 | 1 | rs12493607 | G | C |
| 3 | 30682947 | 0 | rs13093591 | T | C |
| 3 | 30683034 | 0 | rs34771216 | C | G |
| 3 | 46866866 | 1 | rs6796502 | G | A |
| 3 | 63967900 | 1 | rs1053338 | A | G |
| 4 | 106084778 | 1 | rs9790517 | C | T |
| 4 | 106086302 | 0 | rs12641113 | C | T |
| 4 | 106087822 | 0 | rs10022109 | A | G |
| 4 | 175821735 | 0 | rs1319629 | C | T |
| 4 | 175821923 | 0 | rs985261 | C | T |
| 4 | 175822728 | 0 | rs966765 | G | A |
| 4 | 175822759 | 0 | rs6553846 | T | C |
| 4 | 175823392 | 0 | rs12506943 | G | A |
| 4 | 175827456 | 0 | rs13139984 | C | T |
| 4 | 175827730 | 0 | rs13120671 | A | T |
| 4 | 175828287 | 0 | rs7669051 | C | T |
| 4 | 175828408 | 0 | rs7669284 | C | T |
| 4 | 175828505 | 0 | rs7684319 | G | A |
| 4 | 175829485 | 0 | rs10020805 | G | A |
| 4 | 175829510 | 0 | rs10010683 | A | G |
| 4 | 175829561 | 0 | rs10013130 | T | C |
| 4 | 175829693 | 0 | rs10020993 | G | T |
| 4 | 175829805 | 0 | rs9999409 | C | T |
| 4 | 175830130 | 0 | rs28647940 | G | A |
| 4 | 175830257 | 0 | rs10049880 | A | G |
| 4 | 175831761 | 0 | rs72999964 | C | A |
| 4 | 175832937 | 0 | rs7664956 | G | T |
| 4 | 175833091 | 0 | rs9884717 | A | G |
| 4 | 175834588 | 0 | rs72999969 | G | A |
| 4 | 175835660 | 0 | rs4695981 | C | G |
| 4 | 175837416 | 0 | rs28464422 | A | G |
| 4 | 175837642 | 0 | rs28439497 | C | T |
| 4 | 175838941 | 0 | rs10032806 | G | A |
| 4 | 175839285 | 0 | rs4072805 | C | G |
| 4 | 175839432 | 0 | rs1104945 | A | C |
| 4 | 175839725 | 0 | rs4330336 | C | T |
| 4 | 175839727 | 0 | rs9991047 | G | A |
| 4 | 175840903 | 0 | rs7666569 | T | C |
| 4 | 175842495 | 0 | rs28436676 | G | A |
| 4 | 175842979 | 0 | rs28475635 | G | A |
| 4 | 175844215 | 0 | rs200229430 | TTA | T |
| 4 | 175844216 | 0 | rs33957113 | TA | T |
| 4 | 175844270 | 0 | rs28750347 | G | A |
| 4 | 175844531 | 0 | rs28713645 | T | G |
| 4 | 175844585 | 0 | rs28566513 | G | C |
| 4 | 175845869 | 0 | rs6827315 | C | T |
| 4 | 175846110 | 0 | rs6826366 | G | A |
| 4 | 175846320 | 0 | rs6853513 | A | G |
| 4 | 175846426 | 1 | rs6828523 | C | A |
| 4 | 175847527 | 0 | rs9312575 | T | C |
| 4 | 175849984 | 0 | rs9998487 | T | A |
| 5 | 1296255 | 1 | rs3215401 | A | AG |
| 5 | 1297488 | 1 | rs2736108 | C | T |
| 5 | 16187528 | 1 | rs13162653 | G | T |
| 5 | 32567732 | 1 | rs2012709 | C | T |
| 5 | 44662399 | 0 | rs10941677 | G | A |
| 5 | 44662515 | 1 | rs4415084 | C | T |
| 5 | 44666965 | 0 | rs6874055 | T | A |
| 5 | 44706498 | 1 | rs10941679 | A | G |
| 5 | 56031884 | 1 | rs889312 | C | A |
| 5 | 58184061 | 1 | rs10472076 | T | C |
| 5 | 58337481 | 1 | rs1353747 | T | G |
| 5 | 58338437 | 0 | rs1553113 | A | C |
| 5 | 58350588 | 0 | rs2968010 | T | A |
| 5 | 81533735 | 0 | rs6888977 | C | T |
| 5 | 81538046 | 1 | rs7707921 | T | A |
| 5 | 81550043 | 0 | rs6884232 | G | A |
| 5 | 81551659 | 0 | rs1019806 | G | A |
| 5 | 81553815 | 0 | rs4703879 | A | G |
| 5 | 81555328 | 0 | rs2407153 | T | G |
| 5 | 158244083 | 1 | rs1432679 | C | T |
| 6 | 1318878 | 1 | rs11242675 | C | T |
| 6 | 1319005 | 0 | rs11242676 | C | T |
| 6 | 13715303 | 0 | rs24023 | A | G |
| 6 | 13715997 | 0 | rs424001 | C | T |
| 6 | 13716711 | 0 | rs381560 | G | A |
| 6 | 13716723 | 0 | rs381551 | G | A |
| 6 | 13717455 | 0 | rs420874 | T | A |
| 6 | 13717913 | 0 | rs495633 | G | A |
| 6 | 13717932 | 0 | rs495572 | A | C |
| 6 | 13718126 | 0 | rs368512 | G | A |
| 6 | 13718872 | 0 | rs571676 | T | C |
| 6 | 13719129 | 0 | rs371729 | C | T |
| 6 | 13722523 | 1 | rs204247 | G | A |
| 6 | 13723374 | 0 | rs204246 | A | T |
| 6 | 28926220 | 1 | rs9257408 | G | C |
| 6 | 82128386 | 1 | rs17529111 | T | C |
| 6 | 127595786 | 0 | rs2144742 | C | A |
| 6 | 127596782 | 0 | rs6906717 | C | A |
| 6 | 127597591 | 0 | rs9385419 | A | G |
| 6 | 127598619 | 0 | rs6569478 | G | A |
| 6 | 127600630 | 1 | rs2180341 | G | A |
| 6 | 127605898 | 0 | rs4897207 | C | T |
| 6 | 127606160 | 0 | rs2326567 | G | A |
| 6 | 127609691 | 0 | rs9321073 | C | T |
| 6 | 127613966 | 0 | rs3798850 | C | T |
| 6 | 151946629 | 1 | rs12665607 | T | A |
| 6 | 151947757 | 0 | rs74295874 | C | T |
| 6 | 151948366 | 1 | rs2046210 | G | A |
| 6 | 151952002 | 0 | rs9397436 | A | G |
| 6 | 151952332 | 1 | rs9397437 | G | A |
| 6 | 151953765 | 0 | rs9383590 | T | C |
| 6 | 151953859 | 0 | rs9397068 | G | A |
| 6 | 152432902 | 1 | rs910416 | C | T |
| 7 | 91627500 | 0 | rs2299235 | C | T |
| 7 | 91628593 | 0 | rs2018628 | G | T |
| 7 | 91630620 | 1 | rs6964587 | G | T |
| 7 | 91633213 | 0 | rs12540565 | T | G |
| 7 | 91634963 | 0 | rs12539231 | G | A |
| 7 | 91638451 | 0 | rs28399886 | A | G |
| 7 | 91639313 | 0 | rs7455444 | C | T |
| 7 | 91640273 | 0 | rs6465344 | G | A |
| 7 | 91640773 | 0 | rs7785095 | A | T |
| 7 | 91641928 | 0 | rs13245393 | A | G |
| 7 | 91642714 | 0 | rs6944591 | T | C |
| 7 | 91643203 | 0 | rs202142712 | AC | A |
| 7 | 91643219 | 0 | rs6967256 | G | A |
| 7 | 91644070 | 0 | rs28594877 | C | T |
| 7 | 91644553 | 0 | rs7805077 | A | G |
| 7 | 91645152 | 0 | rs10234071 | G | C |
| 7 | 91645265 | 0 | rs10263309 | A | G |
| 7 | 91646198 | 0 | rs7788092 | C | T |
| 7 | 91647390 | 0 | rs28410528 | A | G |
| 7 | 91648341 | 0 | rs2888851 | G | A |
| 7 | 91648744 | 0 | rs13221998 | A | G |
| 7 | 91648939 | 0 | rs7802668 | A | G |
| 7 | 91653851 | 0 | rs13231238 | T | G |
| 7 | 91657116 | 0 | rs147131837 | A | G |
| 7 | 91657994 | 0 | rs6952389 | A | G |
| 7 | 91659150 | 0 | rs17164315 | C | G |
| 7 | 91660053 | 0 | rs10281556 | A | G |
| 7 | 91660225 | 0 | rs10488510 | G | T |
| 7 | 91663266 | 0 | rs13231578 | C | T |
| 7 | 91663364 | 0 | rs7811564 | A | G |
| 7 | 130667121 | 1 | rs4593472 | C | T |
| 7 | 144074929 | 1 | rs720475 | G | A |
| 8 | 29505165 | 0 | rs7465364 | A | G |
| 8 | 29505608 | 0 | rs7845360 | A | T |
| 8 | 29507094 | 0 | rs7463114 | T | C |
| 8 | 29509616 | 1 | rs9693444 | A | C |
| 8 | 36858483 | 1 | rs13365225 | A | G |
| 8 | 76230301 | 1 | rs6472903 | G | T |
| 8 | 76230943 | 0 | rs1511243 | A | G |
| 8 | 76236251 | 0 | rs6472904 | C | A |
| 8 | 76405582 | 0 | rs2977904 | C | T |
| 8 | 76410861 | 0 | rs2926585 | A | G |
| 8 | 76411518 | 0 | rs2926586 | A | T |
| 8 | 76412152 | 0 | rs2943604 | T | A |
| 8 | 76412189 | 0 | rs2977949 | A | C |
| 8 | 76415046 | 0 | rs2977896 | A | T |
| 8 | 76417937 | 1 | rs2943559 | A | G |
| 8 | 76419046 | 0 | rs2943568 | C | A |
| 8 | 76422005 | 0 | rs2977909 | A | T |
| 8 | 117209548 | 1 | rs13267382 | A | G |
| 8 | 128355618 | 1 | rs13281615 | A | G |
| 8 | 128387852 | 1 | rs1562430 | T | C |
| 8 | 129186110 | 0 | rs72722756 | T | C |
| 8 | 129194009 | 0 | rs67397162 | C | T |
| 8 | 129194641 | 1 | rs11780156 | C | T |
| 8 | 129199566 | 0 | rs1016578 | G | A |
| 9 | 110305088 | 0 | rs10759242 | C | A |
| 9 | 110306115 | 1 | rs10759243 | C | A |
| 9 | 110885947 | 0 | rs519679 | C | G |
| 9 | 110886052 | 0 | rs520613 | C | T |
| 9 | 110886254 | 0 | rs522463 | G | T |
| 9 | 110886534 | 0 | rs525142 | G | A |
| 9 | 110886745 | 0 | rs527071 | C | A |
| 9 | 110887106 | 0 | rs648354 | G | A |
| 9 | 110887996 | 0 | rs662694 | C | G |
| 9 | 110888113 | 0 | rs471467 | G | A |
| 9 | 110888260 | 0 | rs472483 | T | C |
| 9 | 110888478 | 1 | rs865686 | G | T |
| 9 | 110888809 | 0 | rs857610 | A | G |
| 10 | 22032942 | 1 | rs7072776 | A | G |
| 10 | 22303789 | 0 | rs7078177 | G | A |
| 10 | 22315843 | 1 | rs11814448 | A | C |
| 10 | 22319508 | 0 | rs12248406 | C | T |
| 10 | 22320581 | 0 | rs11012846 | C | T |
| 10 | 64276964 | 0 | rs34511355 | A | C |
| 10 | 64278181 | 0 | rs10995189 | G | A |
| 10 | 64278682 | 1 | rs10995190 | G | A |
| 10 | 64278874 | 0 | rs10995191 | C | T |
| 10 | 80841148 | 1 | rs704010 | T | C |
| 10 | 114773927 | 1 | rs7904519 | A | G |
| 10 | 114777396 | 0 | rs7918599 | C | T |
| 10 | 114777724 | 0 | rs10885406 | A | G |
| 10 | 114780633 | 0 | rs11196191 | A | C |
| 10 | 114781297 | 0 | rs10787472 | A | C |
| 10 | 114781400 | 0 | rs10787473 | C | A |
| 10 | 114781698 | 0 | rs12258200 | T | C |
| 10 | 114783403 | 0 | rs6585203 | C | G |
| 10 | 123093182 | 0 | rs9420318 | G | A |
| 10 | 123093901 | 1 | rs11199914 | C | T |
| 10 | 123337335 | 1 | rs2981579 | A | G |
| 11 | 1909006 | 1 | rs3817198 | T | C |
| 11 | 65579600 | 0 | rs10896052 | C | A |
| 11 | 65582341 | 0 | rs3892696 | G | C |
| 11 | 65583066 | 1 | rs3903072 | G | T |
| 11 | 69330983 | 0 | rs661204 | G | A |
| 11 | 69331418 | 1 | rs78540526 | C | T |
| 11 | 69331642 | 1 | rs554219 | C | G |
| 11 | 69332670 | 0 | rs657686 | A | G |
| 11 | 129462233 | 1 | rs745382 | A | G |
| 12 | 14413931 | 1 | rs12422552 | G | C |
| 12 | 28155080 | 1 | rs10771399 | A | G |
| 12 | 28174817 | 1 | rs7297051 | C | T |
| 12 | 96027759 | 1 | rs17356907 | A | G |
| 12 | 115835798 | 0 | rs2464264 | G | A |
| 12 | 115835836 | 0 | rs2454399 | T | C |
| 12 | 115836132 | 0 | rs1391721 | T | C |
| 12 | 115836522 | 1 | rs1292011 | A | G |
| 13 | 32968550 | 0 | rs11571815 | G | A |
| 13 | 32968810 | 0 | rs11571818 | T | C |
| 13 | 32972626 | 1 | rs11571833 | A | T |
| 13 | 73811471 | 1 | rs17181761 | A | C |
| 13 | 73813803 | 0 | rs9573140 | A | G |
| 13 | 73814441 | 0 | rs9543287 | C | G |
| 13 | 73814697 | 0 | rs9530173 | A | G |
| 13 | 73957681 | 1 | rs6562760 | A | G |
| 14 | 37132769 | 1 | rs2236007 | G | A |
| 14 | 37135752 | 0 | rs12881240 | C | T |
| 14 | 68660428 | 1 | rs2588809 | T | C |
| 14 | 69034682 | 1 | rs999737 | C | T |
| 14 | 69036127 | 0 | rs17756147 | G | A |
| 14 | 91841069 | 1 | rs941764 | A | G |
| 14 | 93104072 | 1 | rs11627032 | T | C |
| 16 | 52534167 | 1 | rs8051542 | T | C |
| 16 | 52586341 | 1 | rs3803662 | A | G |
| 16 | 52586477 | 0 | rs3803661 | A | G |
| 16 | 53811788 | 0 | rs62033400 | A | G |
| 16 | 53812433 | 0 | rs8063057 | T | C |
| 16 | 53813367 | 1 | rs17817449 | T | G |
| 16 | 53855291 | 1 | rs11075995 | A | T |
| 16 | 80650805 | 1 | rs13329835 | A | G |
| 17 | 29230520 | 1 | rs146699004 | GGT | G |
| 17 | 53048442 | 0 | rs9895808 | C | G |
| 17 | 53048469 | 0 | rs9897447 | T | C |
| 17 | 53048542 | 0 | rs9896044 | C | G |
| 17 | 53048924 | 0 | rs9902687 | G | A |
| 17 | 53049869 | 0 | rs8080491 | T | A |
| 17 | 53049987 | 0 | rs6504948 | T | C |
| 17 | 53050133 | 0 | rs6504949 | T | G |
| 17 | 53053379 | 0 | rs8078550 | T | G |
| 17 | 53054367 | 0 | rs9914732 | C | G |
| 17 | 53054497 | 0 | rs9916642 | T | C |
| 17 | 53054697 | 0 | rs9915832 | G | A |
| 17 | 53054749 | 0 | rs9893306 | A | T |
| 17 | 53055246 | 0 | rs9894529 | A | T |
| 17 | 53056471 | 1 | rs6504950 | G | A |
| 17 | 53056975 | 0 | rs6504951 | A | G |
| 17 | 53057391 | 0 | rs71300611 | C | CTA |
| 17 | 53057747 | 0 | rs9903146 | C | T |
| 17 | 53057764 | 0 | rs9902950 | A | T |
| 17 | 53057865 | 0 | rs9903220 | A | G |
| 17 | 53057893 | 0 | rs9903444 | C | T |
| 17 | 53057914 | 0 | rs9903825 | G | A |
| 17 | 53058676 | 0 | rs28558726 | A | G |
| 17 | 53058807 | 0 | rs16955471 | G | T |
| 17 | 53060033 | 0 | rs9891865 | C | T |
| 17 | 53061075 | 0 | rs1990674 | G | A |
| 17 | 53061622 | 0 | rs9902718 | T | C |
| 17 | 53062903 | 0 | rs10468513 | C | A |
| 17 | 53064550 | 0 | rs56348638 | C | T |
| 17 | 53065807 | 0 | rs7219874 | C | T |
| 17 | 53067993 | 0 | rs8082471 | T | A |
| 17 | 77781387 | 0 | rs745571 | T | C |
| 17 | 77781725 | 1 | rs745570 | A | G |
| 18 | 24332476 | 1 | rs1667550 | A | G |
| 18 | 24570667 | 1 | rs1436904 | T | G |
| 18 | 24571244 | 0 | rs1786612 | C | T |
| 18 | 24571469 | 0 | rs74435363 | C | CAG |
| 18 | 24579856 | 0 | rs1154208 | C | T |
| 18 | 42399590 | 1 | rs6507583 | A | G |
| 19 | 17390291 | 0 | rs4808075 | T | C |
| 19 | 17391328 | 0 | rs10419397 | G | A |
| 19 | 17393925 | 1 | rs56069439 | C | A |
| 19 | 18571141 | 1 | rs4808801 | A | G |
| 19 | 44286513 | 1 | rs3760982 | A | G |
| 19 | 44286660 | 0 | rs3760983 | T | C |
| 19 | 44286762 | 0 | rs3760984 | C | T |
| 19 | 44286982 | 0 | rs11665924 | G | A |
| 19 | 44287234 | 0 | rs5828181 | A | ATG |
| 19 | 44287707 | 0 | rs4802199 | C | T |
| 19 | 44289518 | 0 | rs4802200 | G | A |
| 19 | 44289824 | 0 | rs11669175 | A | G |
| 19 | 44289994 | 0 | rs35710280 | CA | C |
| 19 | 44290013 | 0 | rs4803658 | T | A |
| 20 | 5948227 | 1 | rs16991615 | G | A |
| 21 | 16520832 | 1 | rs2823093 | G | A |
| 22 | 28995704 | 0 | rs185936232 | T | G |
| 22 | 29008888 | 0 | rs186184919 | C | T |
| 22 | 29098375 | 0 | rs191767420 | C | T |
| 22 | 29098376 | 0 | rs182075939 | A | G |
| 22 | 29121087 | 1 | rs17879961 | A | G |
| 22 | 29621477 | 1 | rs132390 | C | T |
| 22 | 40778231 | 1 | rs17001868 | A | C |
| 22 | 40875199 | 1 | rs73167067 | C | G |

In some embodiments, SNPs that are within 50 kilobases of the SNPs in Table 1 may be used as a proxy in the calculation of the PRS. In some embodiments, SNPs that are within 100 kilobases of the SNPs in Table 1 may be used as a proxy in the calculation of the PRS. In some embodiments, SNPs that are within 150 kilobases of the SNPs in Table 1 may be used as a proxy in the calculation of the PRS. In some embodiments, SNPs that are within 200 kilobases of the SNPs in Table 1 may be used as a proxy in the calculation of the PRS. In some embodiments, SNPs that are within 250 kilobases of the SNPs in Table 1 may be used as a proxy in the calculation of the PRS. In some embodiments, SNPs that are within 300 kilobases of the SNPs in Table 1 may be used as a proxy in the calculation of the PRS. In some embodiments, SNPs that are within 350 kilobases of the SNPs in Table 1 may be used as a proxy in the calculation of the PRS. In some embodiments, SNPs that are within 400 kilobases of the SNPs in Table 1 may be used as a proxy in the calculation of the PRS. In some embodiments, SNPs that are within 450 kilobases of the SNPs in Table 1 may be used as a proxy in the calculation of the PRS. In some embodiments, SNPs that are within 500 kilobases of the SNPs in Table 1 may be used as a proxy in the calculation of the PRS.

In some embodiments, SNPs that have a pairwise r²=1.0 with respect to the SNPs in Table 1, may be used as a proxy in the calculation of the PRS. In some embodiments, SNPs that have a pairwise r²=0.9 with respect to the SNPs in Table 1, may be used as a proxy in the calculation of the PRS. In some embodiments, SNPs that have a pairwise r²=0.8 with respect to the SNPs in Table 1, may be used as a proxy in the calculation of the PRS. In some embodiments, SNPs that have a pairwise r²=0.7 with respect to the SNPs in Table 1, may be used as a proxy in the calculation of the PRS. In some embodiments, SNPs that have a pairwise r²=0.6 with respect to the SNPs in Table 1, may be used as a proxy in the calculation of the PRS. In some embodiments, SNPs that have a pairwise r²=0.5 with respect to the SNPs in Table 1, may be used as a proxy in the calculation of the PRS. In some embodiments, SNPs that have a pairwise r²=0.4 with respect to the SNPs in Table 1, may be used as a proxy in the calculation of the PRS. In some embodiments, SNPs that have a pairwise r²=0.3 with respect to the SNPs in Table 1, may be used as a proxy in the calculation of the PRS. In some embodiments, SNPs that have a pairwise r²=0.2 with respect to the SNPs in Table 1, may be used as a proxy in the calculation of the PRS. In some embodiments, SNPs that have a pairwise r²=0.1 with respect to the SNPs in Table 1, may be used as a proxy in the calculation of the PRS.

In some embodiments, the PRS is calculated by a method that comprises: computing an unscaled population risk score according to the equation μ=(1−p)²+2p(1−p)OR+p²OR², wherein i is unscaled population risk, p is a risk allele frequency, and OR is a per-allele odds ratio for each SNP. Next, calculating the adjusted risk values using p according to: 1/μ, when 0 risk alleles are present, OR/μ, when 1 risk allele is present; OR²/μ, when 2 risk alleles are present; and multiplying together the adjusted risk values for each SNP of the at least 50 SNPs to calculate the PRS for a patient based on the patient's observed genotypes.

In some embodiments, if the PRS score is at least 20% greater than the average population risk, the method of treatment includes physician recommended screenings of patients. In further embodiments, these patient screenings include increased frequency of screenings. In still further embodiments, these screenings include, but are not limited to: mammograms, one or more breast magnetic resonance imaging (MRI) scans, one or more clinical breast exams, ultrasound, and taking one or more additional biological samples for genetic testing. In further embodiments the biological samples taken for additional testing include tissue taken from biopsies and blood samples.

In some embodiments, if the PRS score is at least 20% greater than the average population risk, the method of treatment includes the physician recommending surgeries to the patient to remove breast tissue and includes but is not limited to: a prophylactic mastectomy, a mastectomy, and breast conservation surgery.

In some embodiments, if the PRS score is at least 20% greater than the average population risk, the method of treatment includes the physician recommending drug treatments. The types of drugs prescribed in a treatment includes preventative drugs, such as, but are not limited to: raloxifene hydrochloride and tamoxifen citrate.

In some embodiments, if the PRS score is at least 20% greater than the average population risk, the method of treatment includes the physician recommending drug treatments. The types of drugs prescribed in treatment includes drugs, such as, but are not limited to: Abemaciclib, Ado-Trastuzumab Emtansine, Anastrozole, Capecitabine, Cyclophosphamide, Docetaxel, Doxorubicin Hydrochloride, Epirubicin Hydrochloride, Eribulin Mesylate, Everolimus, Exemestane, Fluorouracil Injection, Fulvestrant, Gemcitabine Hydrochloride, Goserelin Acetate, Ixabepilone, Lapatinib Ditosylate, Letrozole, Megestrol Acetate, Methotrexate, Neratinib Maleate, Olaparib, Paclitaxel, Paclitaxel Albumin-stabilized Nanoparticle Formulation, Palbociclib, Pamidronate Disodium, Pertuzumab, Ribociclib, Tamoxifen Citrate, Thiotepa, Toremifene, Trastuzumab, and Vinblastine Sulfate.

In some embodiments, the medical procedure recommended to the patient, as set forth above, is based on the patient having a polygenic risk score that is at least 30% greater than the average population risk. In other embodiments, the medical procedure recommended to the patient, as set forth above, is based on the patient having a polygenic risk score that is at least 40% greater than the average population risk. In other embodiments, the medical procedure recommended to the patient, as set forth above, is based on the patient having a polygenic risk score that is at least 50% greater than the average population risk. In other embodiments, the medical procedure recommended to the patient, as set forth above, is based on the patient having a polygenic risk score that is at least 60% greater than the average population risk.

In some embodiments, the PRS is combined with a score derived from patient history information to calculate an absolute risk to the patient of developing cancer. In some embodiments, the patient history information includes, but is not limited to: age, sex, breast density, birth control, obesity, alcohol use and family breast cancer history.

In one embodiment the Tyrer-Cuzick model is used. As described in Tyrer et al. 2016, and incorporated in its entirety, the Tyrer-Cuzick model is a breast cancer risk score that includes information provided by patients. The model uses information including, but is not limited to: age, a detailed family history of breast and ovarian cancer in first and second degree relatives with age at onset, prior proliferative benign breast disease or atypical hyperplasia, hormone replacement therapy use, height, weight, age at menopause, and parity including age at first child birth. In further embodiments, the information is taken directly from a patient or obtained from the patient's history file, by either the physician or a third party entity given consent to access the file history in order to calculate the score.

In one embodiments, the PRS score is used to independently verify the Tyrer-Cuzick score when recommending medical procedures to a patient.

In another embodiment, the patient history score derived using the Tyrer-Cuzick model is multiplied together with the PRS to calculate an absolute risk known as the Ambry Combined Score.

In one embodiment, the medical procedure recommended to the patient, as set forth above, is based on an Ambry Combined Score calculating an absolute risk to the patient of developing cancer within their lifetime of at least 20%.

In some embodiments, the medical procedure recommended to the patient, as set forth above, is based on the Ambry Combined Score calculating an absolute risk to the patient of developing cancer within their lifetime of at least 30%. In some embodiments, the medical procedure recommended to the patient, as set forth above, is based on the Ambry Combined Score calculating an absolute risk to the patient of developing cancer within their lifetime of at least 40%. In some embodiments, the medical procedure recommended to the patient, as set forth above, is based on the Ambry Combined Score calculating an absolute risk to the patient of developing cancer within their lifetime of at least 50%. In some embodiments, the medical procedure recommended to the patient, as set forth above, is based on the Ambry Combined Score calculating an absolute risk to the patient of developing cancer within their lifetime of at least 60%.

In some embodiments the SNPs are analyzed using next generation sequencing platforms. In further embodiments, the SNPs are sequenced with commercial next generation sequencing probes. In still further embodiments, SNPs are sequenced with commercial next generation sequencing probes that have been either supplemented or augmented based on an experimenters preference in order to improve the ability to collect data and the efficiency at which it is obtained.

In other embodiments, SNPs are analyzed using a variety of techniques including: SNP microarrays, molecular beacons, dynamic allele-specific hybridization, restriction fragment length polymorphism, PCR-based methods, flap endonuclease, 5′-nuclease assays, primer extension, single strand polymorphism, temperature gradient gel electrophoresis, and denaturing high performance liquid chromatography.

In some embodiments, the PRS is calculated from a woman without a pathogenic or likely pathogenic BRCA-1 and/or BRCA-2 gene.

In some embodiments, the PRS is calculated from a woman without pathogenic or likely pathogenic variants of the genes: ATM, BARD1, BLM, BRIP1, CDH1, CHEK2, FANCC, MRE11A, NBN, NF1, PALB2, PTEN, RAD50, RAD51C, RAD51D, STK11, and TP53.

In some embodiments the patient is a woman of Caucasian, non-Ashkenazi Jewish, descent.

In some embodiments the absolute risk indicates a lifetime risk of developing breast cancer up to age 85.

It will be understood that any embodiments from any aspect, where applicable, can be used in combination with other embodiments.

The following non-limiting methods are provided to further illustrate the embodiments of the invention disclosed herein. It should be appreciated by those of skill in the art that the techniques disclosed in the examples that follow represent approaches that have been found to function well in the practice of several embodiments of the invention, and thus be considered to constitute examples of modes for its practice. However, those of skill in the art should, in light of the present disclosure, appreciate that many changes can be made in the specific embodiments that are disclosed and still obtain a like or similar result without departing from the spirit and the scope of the invention.

### EXAMPLES

### Method 1

### Creating the SNP List for Study Samples

A total of 100 SNPs were identified from genome wide association studies presented in the literature as set forth in Table 1. These SNPs were chosen to be used in calculating a polygenic risk score. SNPs from individuals or populations from non-Caucasian and Ashkenazi Jewish descent were excluded from Table 1. Additionally, the SNPs listed in Table 1 were chosen because of they had p-values that were less than or equal to 5×10⁴.

### Method 2

### Criteria for Study Samples

Women were included in the study sample if they were: female, self-reported Caucasian, of non-Ashkenazi Jewish descent, between 18 to 84 years of age at the time of testing, and provided information regarding family history to ordering clinicians.

Women who tested positive for a pathogenic or likely pathogenic with regards to a breast cancer-susceptibility gene (ATM, BARD1, BLM, BRCA1, BRCA2, BRIP1, CDH1, CHEK2, FANCC, MRE11A, NBN, NF1, PALB2, PTEN, RAD50, RAD51C, RAD51D, STK11, TP53) were excluded.

Cases were identified as those with a personal history of breast cancer, and were excluded if clinical history included other cancer primaries. Controls were unaffected with any cancer (not including basal or squamous cell carcinoma); those with a first- or second-degree relative with breast or ovarian cancer were further excluded from analysis.

### Method 3

### Molecular Analysis

Biological samples taken from patients were analyzed by using next generation sequencing molecular analysis was performed using Illumina's NextSeq 500 system.

Sequencing quality for Illumina NextSeq 500 are monitored during the sequencing run, and include visualization of Intensity-vs-Cycle (IVC) plots, and cluster intensity over the duration of the run. Other quality metrics that are evaluated for the entire sequencing run upon completion of sequencing and demultiplexing of the samples include metrics for the % Perfect Index Reads, % of ≥Q30 Bases, and overall Mean Quality Score.

### Method 4

### Statistical Analysis

Samples passing the sequencing quality metrics were fed into a proprietary next generation sequencing data processing pipeline in a parallelized fashion, starting with alignment of sequencing reads to human reference genome build (GRCh37/hg19), followed by variant and genotype calling on the panel genes and the 100 breast cancer-associated SNP positions. Additionally, next generation sequencing coverage is evaluated for all 100 breast cancer associated SNPs for every sample, and any SNPs with no or low coverage (<20×) were excluded from genotype calling, and were not included in downstream statistical analysis.

Next generation sequencing data were examined to assess missing rates for each sample, and each SNP. Samples were excluded if greater than 10 SNPs were missing due to bioinformatics quality control thresholds (n=12; 0.4% of samples). SNP calls were checked for consistency with publically available databases (GRCh37/hg19; Ensembl release 91 {Zerbino et. al.}) and literature-reported reference and risk alleles. SNP allele frequencies were compared among control subjects to those available in the 1000 Genomes EUR population to ensure consistency with the reference population. Hardy Weinberg Equilibrium (HWE) was assessed for all SNPs among controls using R package Hardy-Weinberg (Graffelman et al.).

To assess the assumption of SNP effects consistent with a log additive model, all possible pair-wise SNP*SNP interactions were examined using logistic regression, with a Dickey-Fuller test for the interaction and breast cancer as the outcome. Additional tests were performed for higher-order SNP interactions using logic regression.

Using an approach consistent with prior literature (Dite et al., Mealiffe et al., Cuzick et al., Allman et al.), an SNP-based population-standardized PRS is computed for each patient. Using previously published estimates of the per-allele odds ratio (OR) and risk allele frequency (p) for each SNP, and assuming independent and additive risks on the log OR scale, the unscaled population average risk was calculated as:

μ=(1−p)²+2p(1−p)OR+p²OR² (Equation 1)

Adjusted risk values were then calculated as:

$$\frac{1}{μ} , \frac{OR}{μ} , \frac{{OR}^{2}}{μ} \qquad ( Equation 2 )$$

for the 3 genotypes defined by the number of risk alleles: 0, 1 or 2, respectively. Missing genotypes were assigned a population average risk of 1.0. Adjusted risk values for each SNP were multiplied to compute the overall PRS-associated risk for each individual based on their observed genotypes.

### Method 3

### PRS Validation Assessment

Logistic regression models were used to estimate the ORs for breast cancer by quartile of the PRS, with the 1st quartile category (<25th percentile) as the reference.

The performance of the PRS in predicting breast cancer cases was examined by receiver operating curves (ROC). The area under a receiver operating curve (AUROC) is a graphical way to show the ability of a test's discriminative ability of how good the test in a given clinical situation is. The closer the AUROC is to 1, the better the discriminative ability of the test.

The AUROC was computed using the R package pROC (Robin et al.). R (v.3.3.3) was used for all statistical analyses; all statistical tests were two sided, and p-values <0.05 were considered nominally statistically significant.

### Example 1

### Patient and Case Selection

A total of 3,020 patient samples (1,772 breast cancer cases and 1,248 controls) underwent next generation sequencing. After assessment of quality control and inclusion/exclusion criteria, data from 1,689 breast cancer cases and 1,160 controls were available for analysis. The mean age and standard deviation (mean±SD) at testing for cases and controls was 55.7±11.3 and 47.5±12.9 years, respectively.

### Analysis of Cases

Among cases, the mean±SD age at first diagnosis of breast cancer was 51.0±10.9 years. While 92.0% had at least one close relative (1st, 2nd or 3rd degree) with cancer, 74.8% had a close relative, and 39.7% had at least one first degree relative with breast and/or ovarian cancer. Approximately 21.8% of cases were estrogen receptor negative, and 14.0% had triple negative breast cancer.

The mean±SD SNP call rate, or the proportion of individuals for whom a genotype was successfully determined for a given SNP, was 99.7%/1.1% (range 92.2% to 100.0%). SNP risk allele frequencies (RAF) among controls ranged from 0.8% to 93.5%, and were consistent with the 1000 Genomes non-Finnish EUR population (range: 1.0% to 93.3%; mean±SD absolute difference among SNPs: 0.5%/2.5%, p=0.05).

One SNP was monomorphic in both cases and controls (RAF=0%), as observed in the 1000 Genomes non-Finnish EUR population; the Finnish population carries the risk allele with a frequency of 2.5%, and a frequency of 0.7% has been reported among controls in the literature (Michailidou et al.). Consistent with the findings of previous studies (Mavaddat et al., Mealiffe et al., Milne et al.), there was little to no significant pairwise or high-order interactions among the SNPs after Bonferroni or false discovery rate correction for multiple testing.

### Statistical Analysis

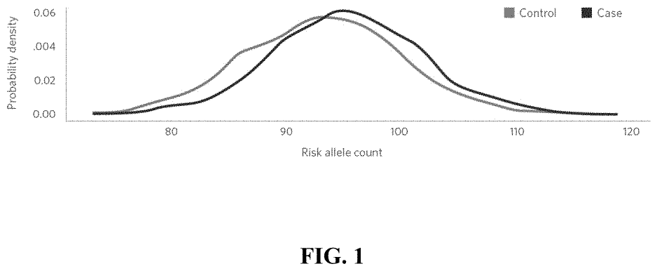

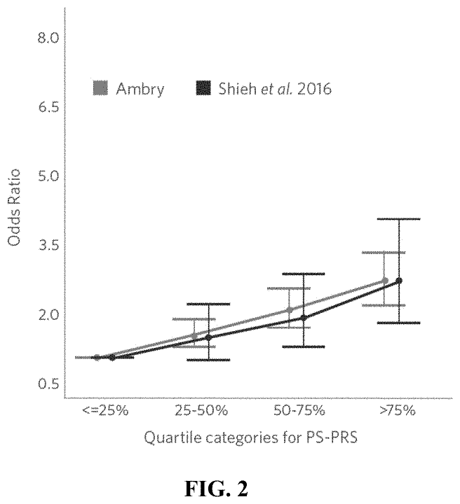

The sum of the risk alleles across the 100 SNPs was approximately normally distributed among cases and controls, and ranged from 75 to 119 and 73 to 111, respectively (mean±SD risk allele count: 95.3±6.5 vs. 93.1±6.7, p<0.0001; FIG. 1). The mean±SD population standardized PRS was significantly higher for cases compared to controls (1.20±0.88 vs. 0.95±0.69, p<0.0001). The OR for breast cancer per standard deviation of the PRS was 1.45 (95% Confidence Interval “CI”: 1.32-1.59). Compared to women in the 1st quartile of PRS, those in the 2nd, 3rd and 4th quartile were 1.51 (95% CI: 1.23-1.87), 2.06 (95% CI: 1.67-2.55) and 2.69 (95% CI: 2.17-3.35) times as likely to have breast cancer (all p<0.0001; FIG. 2).

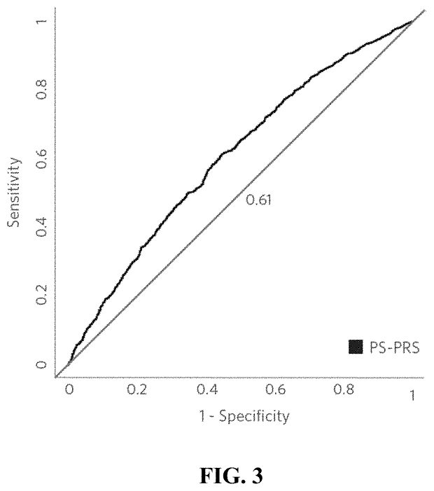

The area under the receiver operating characteristic curve (AUROC) was used to compare discrimination of the models. A maximum AUROC for PRS discrimination of cases and controls was reached at a threshold of 0.83, corresponding to a positive predictive value (PPV) equal to 0.67 and negative predictive value (NPV) equal to 0.50 (AUROC=0.61, 95% CI: 0.59-0.63; FIG. 3).

The results show that overall, the OR per standard deviation reported by this disclosure for the 100-SNP PRS is similar to results obtained from Dite et al. and Shieh et al. Dite et al. reported an OR per standard deviation of the PRS of 1.46 (95% CI: 1.29-1.64). Shieh et al. observed unadjusted ORs for breast cancer of 1.34 (95% CI: 0.90-2.00), 1.76 (95% CI: 1.18-2.62) and 2.54 (95% CI: 1.69-3.82) for the 2nd, 3rd and 4th quartile of PRS compared to the 1st quartile (Shieh et al.). Further, the results also show the validity of the disclosed PRS in predicting breast cancer as demonstrated by a AUROC greater than 0.5. This is consistent with prior reports where AUROC ranged 0.55-0.68 (Mavaddat et al., Dite et al., Mealiffe et al., Shieh et al., Li et al., Sawyer et al., Allman et al., Vachon et al.). The PRS presented in this disclosure therefore has demonstrable performance regarding its ability to predict breast cancer.

### REFERENCES

Global Health Estimates, World Health Organization 2013.
Coleman M P et al., Cancer survival in five continents: a worldwide population-based study (CONCORD). Lancet Oncol., 2008. 9(8): p. 730-56.
Tyrer et al., Models for assessment of breast cancer risk. DiEurope., 2016. p: 54-55.
Easton, D. F., et al., Genome-wide association study identifies novel breast cancer susceptibility loci. Nature, 2007. 447(7148): p. 1087-93.
Michailidou, K., et al., Association analysis identifies 65 new breast cancer risk loci. Nature, 2017. 551(7678): p. 92-94.
Mavaddat, N., et al., Prediction of breast cancer risk based on profiling with common genetic variants. J Natl Cancer Inst, 2015. 107(5).
Dite, G. S., et al., Breast cancer Risk Prediction Using Clinical Models and 77 Independent Risk-Associated SNPs for Women Aged Under 50 Years: Australian Breast cancer Family Registry. Cancer Epidemiol Biomarkers Prev, 2016. 25(2): p. 359-65.
Mealiffe, M. E., et al., Assessment of clinical validity of a breast cancer risk model combining genetic and clinical information. J Natl Cancer Inst, 2010. 102(21): p. 1618-27.
Reeves, G. K., et al., Incidence of breast cancer and its subtypes in relation to individual and multiple low-penetrance genetic susceptibility loci. Jama, 2010. 304(4): p. 426-34.
Shieh, Y., et al., Breast cancer risk prediction using a clinical risk model and polygenic risk score. Breast cancer Res Treat, 2016. 159(3): p. 513-25.
Li, H., et al., Breast cancer risk prediction using a polygenic risk score in the familial setting: a prospective study from the Breast cancer Family Registry and kConFab. Genet Med, 2017. 19(1): p. 30-35.
Sawyer, S., et al., A role for common genomic variants in the assessment of familial breast cancer. J Clin Oncol, 2012. 30(35): p. 4330-6.
Michailidou, K., et al., Large-scale genotyping identifies 41 new loci associated with breast cancer risk. Nat Genet, 2013. 45(4): p. 353-61, 361e1-2.
Michailidou, K., et al., Genome-wide association analysis of more than 120,000 individuals identifies 15 new susceptibility loci for breast cancer. Nat Genet, 2015. 47(4): p. 373-80.
Gold, B., et al., Genome-wide association study provides evidence for a breast cancer risk locus at 6q22.33. Proc Natl Acad Sci USA, 2008. 105(11): p. 43405.
Couch, F. J., et al., Identification of four novel susceptibility loci for oestrogen receptor negative breast cancer. Nat Commun, 2016. 7: p. 11375.
Milne, R. L., et al., Identification of ten variants associated with risk of estrogen-receptor-negative breast cancer. Nat Genet, 2017. 49(12): p. 1767-1778.
Garcia-Closas, M., et al., Genome-wide association studies identify four ER negative-specific breast cancer risk loci. Nat Genet, 2013. 45(4): p. 392-8, 398e1-2.
Fletcher, O., et al., Novel breast cancer susceptibility locus at 9q31.2: results of a genome-wide association study. J Natl Cancer Inst, 2011. 103(5): p. 425-35.
Orr, N., et al., Genome-wide association study identifies a common variant in RAD51B associated with male breast cancer risk. Nat Genet, 2012. 44(11): p. 1182-4.
Lindstrom, S., et al., Genome-wide association study identifies multiple loci associated with both mammographic density and breast cancer risk. Nat Commun, 2014. 5: p. 5303.
Zerbino, D. R., et al., Ensembl 2018. Nucleic Acids Res, 2018. 46(D1): p. D754-d761.
Graffelman, J. and J. M. Camarena, Graphical tests for Hardy-Weinberg equilibrium based on the ternary plot Hum Hered, 2008. 65(2): p. 77-84.
Schwender, H. and K. Ickstadt, Identification of SNP interactions using logic regression. Biostatistics, 2008. 9(1): p. 187-98.
Cuzick, J., et al., Impact of a Panel of 88 Single Nucleotide Polymorphisms on the Risk of Breast cancer in High-Risk Women: Results From Two Randomized Tamoxifen Prevention Trials. J Clin Oncol, 2017. 35(7): p. 743-750.
Allman, R., et al., SNPs and breast cancer risk prediction for African American and Hispanic women. Breast cancer Res Treat, 2015. 154(3): p. 583-9.
Robin, X., et al., pROC: an open-source package for R and S+ to analyze and compare ROC curves. BMC Bioinformatics, 2011. 12(1): p. 77.
Milne, R. L., et al., A large-scale assessment of two-way SNP interactions in breast cancer susceptibility using 46,450 cases and 42,461 controls from the breast cancer association consortium. Hum Mol Genet, 2014. 23(7): p. 193446.
Vachon, C. M., et al., The contributions of breast density and common genetic variation to breast cancer risk. J Natl Cancer Inst, 2015. 107(5).

1. A method for performing a medical procedure on a patient with a potential pre-disposition to cancer comprising:
(a) obtaining a nucleic acid sample from a patient;
(b) assaying the nucleic acid sample obtained from the patient for at least 50 single nucleotide polymorphisms (SNPs) set forth in Table 1, wherein for each SNP in this step, one or more of the following is assayed:
(i) the SNP from Table 1;
(ii) another SNP located within 250 kilobases of the SNP from Table 1;
(iii) another SNP that has a pairwise r²=1.0 with the SNP from Table 1;
(c) calculating a polygenic risk score (PRS) based on the presence or absence of the at least 50 single nucleotide polymorphisms, wherein the polygenic risk score indicates a risk, relative to an average population, that the subject will develop breast cancer; and
(d) performing a medical procedure for the patient based on the PRS.

2. A method for diagnosing a patient with a potential pre-disposition to cancer comprising:
(a) obtaining a nucleic acid sample from a patient;
(b) assaying the nucleic acid sample obtained from the patient for at least 50 single nucleotide polymorphisms (SNPs) set forth in Table 1, wherein for each SNP in this step, one or more of the following is assayed:
(i) the SNP from Table 1;
(ii) another SNP located within 250 kilobases of the SNP from Table 1;
(iii) another SNP that has a pairwise r²=1.0 with the SNP from Table 1;
(c) calculating a polygenic risk score (PRS) based on the presence or absence of the at least 50 single nucleotide polymorphisms, wherein the polygenic risk score indicates a risk, relative to an average population, that the subject will develop breast cancer.

3. The method of claim 1, comprising assaying for at least 65 of the single nucleotide polymorphisms as set forth in Table 1.

4. The method of claim 1, comprising assaying for at least 70 of the single nucleotide polymorphisms as set forth in Table 1.

5. The method of claim 1, comprising assaying for at least 80 of the single nucleotide polymorphisms as set forth in Table 1.

6. The method of claim 1, comprising assaying for at least 95 of the single nucleotide polymorphisms as set forth in Table 1.

7. The method of claim 1, comprising assaying for at least 98 of the single nucleotide polymorphisms as set forth in Table 1.

8. The method of claim 1, wherein the assaying is performed by using a next-generation sequencing platform to detect the single nucleotide polymorphisms in the nucleic acid sample.

9. The method of claim 1, wherein the assaying is performed by microarrays, enzymatic methods, and chromatographic separation techniques.

10. The method of claim 1, wherein calculating the PRS comprises:
calculating an unscaled population risk score for each SNP according to:
μ=(1−p)²+2p(1−p)OR+p²OR², wherein μ is unscaled population risk, p is a risk allele frequency, and OR is a per-allele odds ratio for each SNP;
computing adjusted risk values using p according to:
1/μ, when 0 risk alleles are present;
OR/μ, when 1 risk allele is present;
OR²/μ, when 2 risk alleles are present; and
multiplying together the adjusted risk values for each SNP of the at least 50 SNPs to calculate the PRS for a patient based on the patient's observed genotypes.

11. The method of claim 10, further comprising weighting a patient clinical history score with the PRS score for use in recommending the medical procedure.

12. The method of claim 11, wherein a patient's clinical history includes information regarding age, sex, and family breast cancer history.

13. The method of claim 11, wherein the patient's clinical history score is derived using the Tyrer-Cuzick model.

14. The method of claim 11, wherein the PRS score is multiplied by the patient's clinical history score to calculate an absolute risk.

15. The method of claim 1, wherein the medical procedure comprises performing one or more additional patient screening, administering one or more drug therapies, performing one or more surgeries or any combination thereof.

16. The method of claim 15, wherein additional patient screening include one or more mammograms, one or more breast magnetic resonance imaging (MRI) scans, one or more clinical breast exams, and taking one or more additional biological samples for genetic testing.

17. The method of claim 1, wherein the performing the medical procedure to the patient is based on the patient having a polygenic risk score that is at least 20% greater than the average population risk.

18. The method of claim 1, wherein the performing the medical procedure to the patient is based on an Ambry Combined Score, wherein the Ambry Combined Score is calculated by multiplying the PRS score together with a patient history score derived from the Tyrer-Cuzick model, and wherein the Ambry Combined Score calculates an absolute risk to the patient of developing breast cancer within their lifetime of at least 20%.

19. The method of claim 1, wherein the patient is a woman of Caucasian, non-Ashkenazi Jewish, descent.

20. The method of claim 14, wherein the absolute risk indicates a lifetime risk of developing breast cancer up to age 85.
